(19) United States
(12) Patent Application Publication
(10) Pub. No.: US 2021/0220586 A1
(43) Pub. Date: Jul. 22, 2021

(54) METHOD AND APPARATUS FOR PULSATILE DELIVERY OF NITRIC OXIDE

(71) Applicant: BELLEROPHON THERAPEUTICS, Warren, NJ (US)

(72) Inventors: Parag SHAH, Morristown, NJ (US); Martin DEKKER, Califon, NJ (US); William LEONARD, Basking Ridge, NJ (US); Donatas ZUZEVICIUS, Landing, NJ (US)

(73) Assignee: BELLEROPHON THERAPEUTICS, Warren, NJ (US)

(21) Appl. No.: 17/055,365

(22) PCT Filed: May 17, 2019
(86) PCT No.: PCT/US2019/032887

§ 371 Date: Nov. 13, 2020

Related U.S. Application Data
(60) Provisional application No. 62/672,867, filed on May 17, 2018.

(51) Int. Cl. A61M 16/00; A61M 16/10; A61M 16/06; A61M 15/08; A61M 11/00; A61M 16/12

(52) U.S. Cl. CPC A61M 16/024; A61M 16/1005; A61M 16/0672; A61M 15/08; A61M 11/00; A61M 2202/0208; A61M 2202/0275; A61M 2230/42; A61M 2205/50; A61M 2205/12; A61M 16/12

(57) ABSTRACT

Described are methods for providing a pulsed dose of nitric oxide during at least the first two-thirds of total inspiratory time in each single breath for a therapeutically relevant period of time.

### CROSS-REFERENCE TO RELATED APPLICATIONS

This international PCT application claims the benefit of U.S. provisional application No. 62/672,867, filed on May 17, 2018, which is hereby incorporated by reference in its entirety.

### FIELD OF THE INVENTION

The present application relates generally to apparatus and methods for administration of nitric oxide, in particular, pulsatile delivery of nitric oxide to patients in need of therapeutic treatment.

### BACKGROUND OF THE INVENTION

Nitric oxide (NO) is a gas that, when inhaled, acts to dilate blood vessels in the lungs, improving oxygenation of the blood and reducing pulmonary hypertension. Because of this, nitric oxide is provided as a therapeutic gas in the inspiratory breathing phase for patients who experience shortness of breath (dyspnea) due to a disease state, for example, pulmonary arterial hypertension (PAH), chronic obstructive pulmonary disease (COPD), combined pulmonary fibrosis and emphysema (CPFE), cystic fibrosis (CF), idiopathic pulmonary fibrosis (IPF), emphysema, interstitial lung disease (ILD), chronic thromboembolic pulmonary hypertension (CTEPH), chronic high altitude sickness, or other lung disease.

While NO may be therapeutically effective when administered under the appropriate conditions, it can also become toxic if not administered correctly. NO reacts with oxygen to form nitrogen dioxide (NO₂), and NO₂ can be formed when oxygen or air is present in the NO delivery conduit. NO₂ is a toxic gas which may cause numerous side effects, and the Occupational Safety & Health Administration (OSHA) provides that the permissible exposure limit for general industry is only 5 ppm. Thus, it is desirable to limit exposure to NO₂ during NO therapy.

Effective dosing of NO is based on a number of different variables, including quantity of drug and the timing of delivery. Several patents have been granted relating to NO delivery, including U.S. Pat. Nos. 7,523,752; 8,757,148; 8,770,199; and 8,803,717, and a Design Patent D701,963 for a design of an NO delivery device, all of which are herein incorporated by reference. Additionally, there are pending applications relating to delivery of NO, including US2013/0239963 and US2016/0106949, both of which are herein incorporated by reference. Even in view of these patents and pending publications, there is still a need for methods and apparatuses that deliver NO in a precise, controlled manner, so as to maximize the benefit of a therapeutic dose and minimize the potentially harmful side effects.

### SUMMARY OF THE INVENTION

In an embodiment of the present invention, a method of administering a dose of nitric oxide is described. In an embodiment of the invention, at least a single pulse dose is administered to a patient and is therapeutically effective to treat or alleviate symptoms of a pulmonary disease. In an embodiment of the invention, an aggregate of two or more pulse doses is therapeutically effective to treat or alleviate symptoms of a pulmonary disease.

In an embodiment of the invention, nitric oxide is delivered on a periodic basis for a minimum of five minutes per day to twenty-four hours per day. In an embodiment of the invention, the nitric oxide may be delivered at the convenience of the patient, for example, over a period of time while sleeping. In an embodiment of the invention, administration of pulses of nitric oxide may be evenly or unevenly spaced over a time period (e.g., ten minutes, one hour or for twenty-four hours). In another embodiment, administration of a therapeutically effective dose of nitric oxide may be continuous for a fixed period of time.

In one embodiment, a method comprises detecting a breath pattern in a patient. In an embodiment of the invention, the breath pattern includes the total inspiratory time (e.g., the time duration of a single inspiration of a patient). In an embodiment of the invention, the breath pattern is detected using a device comprising a breath sensitivity control. In an embodiment of the invention, the breath pattern is correlated with an algorithm to calculate the timing of administration of a dose of nitric oxide. In an embodiment of the present invention, the volume of nitric oxide containing gas necessary for administration of an amount of nitric oxide on a per pulse basis is calculated. In an embodiment, the nitric oxide is delivered to the patient in a pulsatile manner over a portion of a total inspiratory time.

In an embodiment of the invention, nitric oxide doses are delivered to the patient over a period of time sufficient to deliver a therapeutic dose of nitric oxide to the patient. In an embodiment of the invention, the device calculates the total time sufficient to deliver a therapeutic dose of nitric oxide to the patient. In an embodiment of the invention, the total time required for a therapeutic dose of nitric oxide to be delivered to the patient is at least partially dependent upon the breath pattern of said patient.

In an embodiment of the invention, nitric oxide is delivered during the first third of the total inspiratory time. In an embodiment, nitric oxide is delivered during the first half of the total inspiratory time. In an embodiment, nitric oxide is delivered during the first two-thirds of the total inspiratory time.

In an embodiment of the invention, at least fifty percent (50%) of the nitric oxide dose is delivered during the first third of the total inspiratory time. In an embodiment of the invention, at least seventy percent (70%) of the nitric oxide dose is delivered to the patient during the first half of the total inspiratory time. In an embodiment, at least ninety percent (90%) of the nitric oxide dose is delivered to the patient during the first two-thirds of the total inspiratory time. In an embodiment of the invention, at least ninety percent (90%) of the nitric oxide dose is delivered to the patient during the first third of the total inspiratory time. In an embodiment of the invention, all of the nitric oxide dose is delivered to the patient during the first half of the total inspiratory time.

In an embodiment of the invention, the breath sensitivity control on the device is adjustable. In an embodiment of the invention, the breath sensitivity control is fixed. In an embodiment of the invention, the breath sensitivity control is adjustable from a range of least sensitive to most sensitive, whereby the most sensitive setting is more sensitive at detecting breaths than the least sensitive setting.

In an embodiment of the invention, a method for treating or alleviating symptoms of a cardiopulmonary disease is described. In an embodiment of the invention, the method comprises detecting a breath pattern in a patient using a device comprising a breath sensitivity control. In an embodiment of the invention, the breath pattern comprises a measurement of total inspiratory time. In an embodiment of the invention, the breath pattern is correlated with an algorithm to calculate the timing of administration of a dose of nitric oxide. In an embodiment of the invention, at least fifty percent (50%) of the dose of nitric oxide is delivered over the first third of the total inspiratory time. In an embodiment of the invention, at least seventy percent (70%) of the dose of nitric oxide is delivered to the patient over the first half of the total inspiratory time. In an embodiment of the invention, at least ninety percent (90%) of the dose of nitric oxide is delivered over the first two-thirds of the total inspiratory time.

In an embodiment of the invention, the device calculates the total time needed to deliver a therapeutically effective amount of nitric oxide to a patient. In an embodiment of the invention, the total time needed to deliver a therapeutically effective amount of nitric oxide is dependent upon one or more of a breath pattern, concentration of nitric oxide in a gas to be delivered to a patient, volume of a pulse dose, and duration of a pulse.

In an embodiment of the invention, the pulmonary disease is selected from idiopathic pulmonary fibrosis (IPF), pulmonary arterial hypertension (PAH), chronic obstructive pulmonary disease (COPD), combined pulmonary fibrosis and emphysema (CPFE), cystic fibrosis (CF), emphysema, interstitial lung disease (ILD), chronic thromboembolic pulmonary hypertension (CTEPH), chronic high altitude sickness, or other lung disease. In an embodiment of the invention, the cardiopulmonary disease is pulmonary hypertension associated with other pulmonary diseases such as Group I-V pulmonary hypertension (PH).

In an embodiment of the invention, a programmable device for delivering a dose of nitric oxide is described. In an embodiment of the invention, the device comprises a nasal delivery portion, a drug cartridge comprising nitric oxide, an oxygen source, a breath sensitivity portion to detect breath patterns in the patient, a breath detection algorithm for determining the dose of nitric oxide delivered to the patient, and a portion for administering the dose of nitric oxide to the patient through a series of pulses that correlate with the inspiratory portion of the breath pattern. In an embodiment of the invention, the breath sensitivity portion of the device comprises an adjustable or fixed breath sensitivity setting. In an embodiment of the invention, the nasal delivery portion is a nasal cannula, a face mask, an atomizer, or a nasal inhaler. In an embodiment of the invention, the breath detection algorithm uses a threshold sensitivity and a slope algorithm. In an embodiment of the invention, the slope algorithm counts a breath as detected when the rate of pressure drop reaches a threshold level.

Various embodiments are listed above and will be described in more detail below. It will be understood that the embodiments listed may be combined not only as listed below, but in other suitable combinations in accordance with the scope of the invention.

The foregoing has outlined rather broadly certain features and technical advantages of the present invention. It should be appreciated by those skilled in the art that the specific embodiments disclosed may be readily utilized as a basis for modifying or designing other structures or processes within the scope present invention. It should also be realized by those skilled in the art that such equivalent constructions do not depart from the spirit and scope of the invention as set forth in the appended claims.

### BRIEF DESCRIPTION OF THE DRAWINGS

The foregoing summary, as well as the following detailed description of the invention, will be better understood when read in conjunction with the appended drawings.

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a graph demonstrating a single measurement of a breath.

FIG. 2 is a graph demonstrating measurement of a delivered pulse of nitric oxide to a patient according to the present invention.

FIG. 3 is a graph demonstrating detection of breaths as a percentage of nitric oxide delivery over total inspiratory time. The dotted line represents a breath sensitivity setting of 8 of 10 (e.g., 80% of maximum sensitivity) on Embodiment 1, the solid line represents a breath sensitivity setting of 10 of 10 (e.g., maximum sensitivity) on Embodiment 1, and the dashed line represents a fixed breath sensitivity setting of 10 on Embodiment 2. The dashed line demonstrates that about 93% of the nitric oxide dose is delivered during the first 33% (or first third) of total inspiratory time, and 100% of the nitric oxide dose is delivered during the first 50% (or first half) of total inspiratory time. The solid line demonstrates that about 62% of the nitric oxide dose is delivered during the first 33% (or first third) of total inspiratory time, about 98% is delivered during the first 50% (or first half) of total inspiratory time, and 100% is delivered during the first 67% (or first two-thirds) of total inspiratory time. The dotted line demonstrates that about 17% of the nitric oxide dose is delivered during the first 33% (or first third) of total inspiratory time, about 72% is delivered during the first 50% (or first half) of total inspiratory time, and about 95% during the first 67% (or first two-thirds) of total inspiratory time.

FIG. 4 depicts the combined results described in FIG. 3.

FIGS. 5A and 5B depict an algorithm for breath detection and delivery of nitric oxide. FIG. 5A demonstrates a threshold algorithm. FIG. 5B demonstrates a slope algorithm.

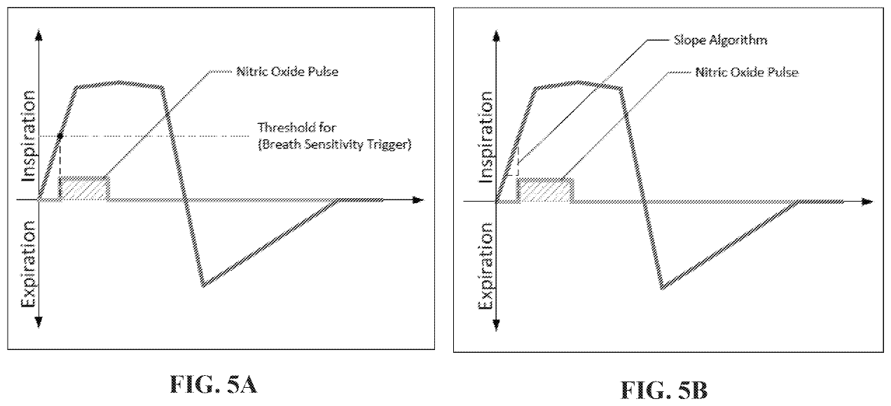

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

Before describing several exemplary embodiments of the invention, it is to be understood that the invention is not limited to the details of construction or process steps set forth in the following description. The invention is capable of other embodiments and of being practiced or being carried out in various ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

### Definitions

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The disease state of “interstitial lung disease” or “ILD” shall include all subtypes of ILD, including, but not limited to, idiopathic interstitial pneumonia (IIP), chronic hypersensitivity pneumonia, occupational or environmental lung disease, idiopathic pulmonary fibrosis (IPF), non-IPF IIPs, granulomoutus (e.g., sarcoidosis), connective tissue disease related ILD, and other forms of ILD.

When ranges are used herein to describe an aspect of the present invention, for example, dosing ranges, amounts of a component of a formulation, etc., all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

With respect to the present invention, in certain embodiments, a dose of a gas (e.g., NO) is administered in a pulse to a patient during an inspiration by the patient. It has been surprisingly discovered that nitric oxide delivery can be precisely and accurately delivered within the first two-thirds of total breath inspiration time and the patient obtains benefits from such delivery. Such delivery minimizes loss of drug product and risk of detrimental side effects increases the efficacy of a pulse dose which in turn results in a lower overall amount of NO that needs to be administered to the patient in order to be effective. Such delivery is useful for the treatment of various diseases, such as but not limited to idiopathic pulmonary fibrosis (IPF), pulmonary arterial hypertension (PAH), including Groups I-V pulmonary hypertension (PH), chronic obstructive pulmonary disease (COPD), combined pulmonary fibrosis and emphysema (CPFE), cystic fibrosis (CF), emphysema, interstitial lung disease (ILD), chronic thromboembolic pulmonary hypertension (CTEPH), chronic high altitude sickness, or other lung disease, and is also useful as an antimicrobial, for example, in treating pneumonia.

Such precision has further advantages in that only portions of the poorly ventilated lung area is exposed to NO. Hypoxia and issues with hemoglobin may also be reduced with such pulsed delivery, while NO₂ exposure is also more limited.

### A Device of the Present Invention

In certain embodiments, the present invention includes a device, e.g. a programmable device for delivering a dose of a gas (e.g., nitric oxide) to a patient in need. The device can include a delivery portion, a drug cartridge including a compressed gas for delivery to a patient, a breath sensitivity portion to detect a breath pattern in patient comprising a breath sensitivity setting, at least one breath detection algorithm for determining when to administer the compressed gas to the patient and a portion for administering the dose of nitric oxide to the patient through a series of one or more pulses.

In certain embodiments, the drug cartridge is replaceable.

In certain embodiments, the delivery portion includes one or more of a nasal cannula, a face mask, an atomizer, and a nasal inhaler. In certain embodiments, the delivery portion can further include a second delivery portion to permit the simultaneous administration of one or more other gases (e.g, oxygen) to a patient.

In certain embodiments, and as detailed elsewhere herein, the device includes an algorithm wherein the algorithm uses one or both of a threshold sensitivity and a slope algorithm, wherein the slope algorithm detects a breath when the rate of pressure drop reaches a predetermined threshold.

In an embodiment of the invention, mechanically, a pulse dose of a gas can reduce, if not eliminate, venturi effects which would normally create problems for other gas sensors. For example, in the absence of the pulse doses of the present invention, O₂ back pressure sensors may override delivery of O₂ when O₂ is administered simultaneously with another gas such as NO.

### Breath Patterns, Detection and Triggers

Breath patterns vary based on the individual, time of day, level of activity, and other variables; thus it is difficult to predetermine a breath pattern of an individual. A delivery system that delivers therapeutics to a patient based on breath pattern, then, should be able to handle a range of potential breath patterns in order to be effective.

In certain embodiments, the patient or individual can be any age, however, in more certain embodiments the patient is sixteen years of age or older.

In an embodiment of the invention, the breath pattern includes a measurement of total inspiratory time, which as used herein is determined for a single breath. However, depending on context “total inspiratory time” can also refer to a summation of all inspiratory times for all detected breaths during a therapy. Total inspiratory time may be observed or calculated. In another embodiment, total inspiratory time is a validated time based on simulated breath patterns.

In an embodiment of the invention, breath detection includes at least one and in some embodiments at least two separate triggers functioning together, namely a breath level trigger and/or a breath slope trigger.

In an embodiment of the invention, a breath level trigger algorithm is used for breath detection. The breath level trigger detects a breath when a threshold level of pressure (e.g., a threshold negative pressure) is reached upon inspiration.

In an embodiment of the invention, a breath slope trigger detects breath when the slope of a pressure waveform indicates inspiration. The breath slope trigger is, in certain instances, more accurate than a threshold trigger, particularly when used for detecting short, shallow breaths.

In an embodiment of the invention, a combination of these two triggers provides overall a more accurate breath detection system, particularly when multiple therapeutic gases are being administered to a patient simultaneously.

In an embodiment of the invention, the breath sensitivity control for detection of either breath level and/or breath slope is fixed. In an embodiment of the invention, the breath sensitivity control for detection of either breath level or breath slope is adjustable or programmable. In an embodiment of the invention, the breath sensitivity control for either breath level and/or breath slope is adjustable from a range of least sensitive to most sensitive, whereby the most sensitive setting is more sensitive at detecting breaths than the least sensitive setting.

In certain embodiments where at least two triggers are used, the sensitivity of each trigger is set at different relative levels. In one embodiment where at least two triggers are used, one trigger is set a maximum sensivity and another trigger is set at less than maximum sensitivity. In one embodiment where at least two triggers are used and where one trigger is a breath level trigger, the breath level trigger is set at maximum sensivity.

Oftentimes, not every inhalation/inspiration of a patient is detected to then be classified as an inhalation/inspiration event for the administration of a pulse of gas (e.g., NO). Errors in detection can occur, particularly when multiple gases are being administered to a patient simultaneously, e.g., NO and oxygen combination therapies.

Embodiments of the present invention, and in particular an embodiment which incorporates a breath slope trigger alone or in combination with another trigger, can maximize the correct detection of inspiration events to thereby maximize the effectiveness and efficiency of a therapy while also minimizing waste due to misidentification or errors in timing.

In certain embodiments, greater than 50% of the total number of inspirations of a patient over a timeframe for gas delivery to the patient are detected. In certain embodiments, greater than 75% of the total number of inspirations of a patient are detected. In certain embodiments, greater than 90% of the total number of inspirations of a patient are detected. In certain embodiments, greater than 95% of the total number of inspirations of a patient are detected. In certain embodiments, greater than 98% of the total number of inspirations of a patient are detected. In certain embodiments, greater than 99% of the total number of inspirations of a patient are detected. In certain embodiments, 75% to 100% of the total number of inspirations of a patient are detected.

Dosages and Dosing Regimens

In an embodiment of the invention, nitric oxide delivered to a patient is formulated at concentrations of about 3 to about 18 mg NO per liter, about 6 to about 10 mg per liter, about 3 mg NO per liter, about 6 mg NO per liter, or about 18 mg NO per liter. The NO may be administered alone or in combination with an alternative gas therapy. In certain embodiments, oxygen (e.g., concentrated oxygen) can be administered to a patient in combination with NO.

In an embodiment of the present invention, a volume of nitric oxide is administered (e.g., in a single pulse) in an amount of from about 0.350 mL to about 7.5 mL per breath. In some embodiments, the volume of nitric oxide in each pulse dose may be identical during the course of a single session. In some embodiments, the volume of nitric oxide in some pulse doses may be different during a single timeframe for gas delivery to a patient. In some embodiments, the volume of nitric oxide in each pulse dose may be adjusted during the course of a single timeframe for gas delivery to a patient as breath patterns are monitored. In an embodiment of the invention, the quantity of nitric oxide (in ng) delivered to a patient for purposes of treating or alleviating symptoms of a pulmonary disease on a per pulse basis (the “pulse dose”) is calculated as follows and rounded to the nearest nanogram value:

Dose ug/kg-IBW/hr×Ideal body weight in kg (kg-IBW)×((1 hr/60 min)/(respiratory rate (bpm))×(1,000 ng/ug).

As an example, Patient A at a dose of 100 ug/kg IBW/hr has an ideal body weight of 75 kg, has a respiratory rate of 20 breaths per minute (or 1200 breaths per hour):

100 ug/kg-IBW/hr×75 kg×(1 hr/1200 breaths)×(1,000 ng/ug)=6250 ng per pulse

In certain embodiments, the 60/respiratory rate (ms) variable may also be referred to as the Dose Event Time. In another embodiment of the invention, a Dose Event Time is 1 second, 2 seconds, 3 seconds, 4 seconds, 5 seconds, 6 seconds, 7 seconds, 8 seconds, 9 seconds, or 10 seconds.

In an embodiment of the invention, a single pulse dose provides a therapeutic effect (e.g., a therapeutically effective amount of NO) to the patient. In another embodiment of the invention, an aggregate of two or more pulse doses provides a therapeutic effect (e.g., a therapeutically effective amount of NO) to the patient.

In an embodiment of the invention, at least about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 625, about 650, about 675, about 700, about 750, about 800, about 850, about 900, about 950, or about 1000 pulses of nitric oxide is administered to a patient every hour.

In an embodiment of the invention, a nitric oxide therapy session occurs over a timeframe. In one embodiment, the timeframe is at least about 1 hour, about 2 hours, about 3 hours, about 4 hours, about 5 hours, about 6 hours, about 7 hours, about 8 hours, about 9 hours, about 10, hours, about 11 hours, about 12 hours, about 13 hours, about 14 hours, about 14 hours, about 15 hours, about 16 hours, about 17 hours, about 18 hours, or about 24 hours per day.

In an embodiment of the invention, a nitric oxide treatment is administered for a timeframe of a minimum course of treatment. In an embodiment of the invention, the minimum course of treatment is about 10 minutes, about 15 minutes, about 20 minutes, about 30 minutes, about 40 minutes, about 50 minutes, about 60 minutes, about 70 minutes, about 80 minutes, or about 90 minutes. In an embodiment of the invention, the minimum course of treatment is about 1 hour, about 2 hours, about 3 hours, about 4 hours, about 5 hours, about 6 hours, about 7 hours, about 8 hours, about 9 hours, about 10, hours, about 11 hours, about 12 hours, about 13 hours, about 14 hours, about 14 hours, about 15 hours, about 16 hours, about 17 hours, about 18 hours, or about 24 hours. In an embodiment of the invention, the minimum course of treatment is about 1, about 2, about 3, about 4, about 5, about 6, or about 7 days, or about 1, about 2, about 3, about 4, about 5, about 6, about 7, or about 8 weeks, or about 1, about 2, about 3, about 4, about 5, about 6, about 7, about 8, about 9, about 10, about 11, about 12, about 18, or about 24 months.

In an embodiment of the invention, a nitric oxide treatment session is administered one or more times per day. In an embodiment of the invention, nitric oxide treatment session may be once, twice, three times, four times, five times, six times, or more than six times per day. In an embodiment of the invention, the treatment session may be administered once a month, once every two weeks, once a week, once every other day, daily, or multiple times in one day.

### Timing of a Pulse of NO

In an embodiment of the invention, the breath pattern is correlated with an algorithm to calculate the timing of administration of a dose of nitric oxide.

The precision of detection of an inhalation/inspiration event also permits the timing of a pulse of gas (e.g., NO) to maximize its efficacy by administering gas at a specified time frame of the total inspiration time of a single detected breath.

In an embodiment of the invention, at least fifty percent (50%) of the pulse dose of a gas is delivered over the first third of the total inspiratory time of each breath. In an embodiment of the invention, at least sixty percent (60%) of the pulse dose of a gas is delivered over the first third of the total inspiratory time. In an embodiment of the invention, at least seventy-five percent (75%) of the pulse dose of a gas is delivered over the first third of the total inspiratory time for each breath. In an embodiment of the invention, at least eighty-five (85%) percent of the pulse dose of a gas is delivered over the first third of the total inspiratory time for each breath. In an embodiment of the invention, at least ninety percent (90%) of the pulse dose of a gas is delivered over the first third of the total inspiratory time. In an embodiment of the invention, at least ninety-two percent (92%) of the pulse dose of a gas is delivered over the first third of the total inspiratory time. In an embodiment of the invention, at least ninety-five percent (95%) of the pulse dose of a gas is delivered over the first third of the total inspiratory time. In an embodiment of the invention, at least ninety-nine (99%) of the pulse dose of a gas is delivered over the first third of the total inspiratory time. In an embodiment of the invention, 90% to 100% of the pulse dose of a gas is delivered over the first third of the total inspiratory time.

In an embodiment of the invention, at least seventy percent (70%) of the pulse dose is delivered to the patient over the first half of the total inspiratory time. In yet another embodiment, at least seventy-five percent (75%) of the pulse dose is delivered to the patient over the first half of the total inspiratory time. In an embodiment of the invention, at least eighty percent (80%) of the pulse dose is delivered to the patient over the first half of the total inspiratory time. In an embodiment of the invention, at least 90 percent (90%) of the pulse dose is delivered to the patient over the first half of the total inspiratory time. In an embodiment of the invention, at least ninety-five percent (95%) of the pulse dose is delivered to the patient over the first half of the total inspiratory time. In an embodiment of the invention, 95% to 100% of the pulse dose of a gas is delivered over the first half of the total inspiratory time

In an embodiment of the invention, at least ninety percent (90%) of the pulse dose is delivered over the first two-thirds of the total inspiratory time. In an embodiment of the invention, at least ninety-five percent (95%) of the pulse dose is delivered over the first two-thirds of the total inspiratory time. In an embodiment of the invention, 95% to 100% of the pulse dose is delivered over the first two-thirds of the total inspiratory time.

When aggregated, administration of a number of pulse doses over a therapy session/timeframe can also meet the above ranges. For example, when aggregated greater than 95% of all the pulse doses administered during a therapy session were administered over the first two thirds of all of the inspiratory times of all of the detected breaths. In higher precision embodiments, when aggregated greater than 95% of all the pulse doses administered during a therapy session were administered over the first third of all of the inspiratory times of all of the detected breaths.

Given the high degree of precision of the detection methodologies of the present invention, a pulse dose can be administered during any specified time window of an inspiration. For example, a pulse dose can be administered targeting the first third, middle third or last third of a patient's inspiration. Alternatively, the first half or second half of an inspiration can be targeted for pulse dose administration. Further, the targets for administration may vary. In one embodiment, the first third of an inspiration time can be targeted for one or a series of inspirations, where the second third or second half may be targeted for one or a series of subsequent inspirations during the same or different therapy session. Alternatively, after the first quarter of an inspiration time has elapsed the pulse dose begins and continues for the middle half (next two quarters) and can be targeted such that the pulse dose ends at the beginning of the last quarter of inspiration time. In some embodiments, the pulse may be delayed by 50, 100, 150, 200, 250, 300, 350, 400, 450, 500, 550, 600, 650, 700, or 750 milliseconds (ms) or a range from about 50 to about 750 milliseconds, from about 50 to about 75 milliseconds, from about 100 to about 750 milliseconds, or from about 200 to about 500 milliseconds.

The utilization of a pulsed dose during inhalation reduces the exposure of poorly ventilated areas of the lung and alveoli from exposure to a pulsed dose gas, e.g., NO. In one embodiment, less than 5% of poorly ventilated (a) areas of the lung or (b) alveoli are exposed to NO. In one embodiment, less than 10% of poorly ventilated (a) areas of the lung or (b) alveoli are exposed to NO. In one embodiment, less than 15% of poorly ventilated (a) areas of the lung or (b) alveoli are exposed to NO. In one embodiment, less than 20% of poorly ventilated (a) areas of the lung or (b) alveoli are exposed to NO. In one embodiment, less than 25% of poorly ventilated (a) areas of the lung or (b) alveoli are exposed to NO. In one embodiment, less than 30% of poorly ventilated (a) areas of the lung or (b) alveoli are exposed to NO. In one embodiment, less than 50% of poorly ventilated (a) areas of the lung or (b) alveoli are exposed to NO. In one embodiment, less than 60% of poorly ventilated (a) areas of the lung or (b) alveoli are exposed to NO. In one embodiment, less than 70% of poorly ventilated (a) areas of the lung or (b) alveoli are exposed to NO. In one embodiment, less than 80% of poorly ventilated (a) areas of the lung or (b) alveoli are exposed to NO. In one embodiment, less than 90% of poorly ventilated (a) areas of the lung or (b) alveoli are exposed to NO.

While preferred embodiments of the invention are shown and described herein, such embodiments are provided by way of example only and are not intended to otherwise limit the scope of the invention. Various alternatives to the described embodiments of the invention may be employed in practicing the invention.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Determination of Precise Breath Sensitivity for Appropriate Trigger/Arming Thresholds

A device using a threshold algorithm to detect breaths was used in this Example (Embodiment 1). A threshold algorithm detects breaths using pressure; that is a pressure drop below a certain threshold must be met upon inspiration to detect and count a breath. That pressure threshold can be modified as a result of varying the detection sensitivity of the Embodiment 1 device. Several breath sensitivity settings were tested in the present Example. Settings from 1 to 10 were tested, with 1 being the least sensitive and 10 being the most sensitive. The trigger threshold, shown in cm H₂O, is the threshold level at which nitric oxide is delivered. The arming threshold, also shown in cm H₂O, is the threshold level at which the device is armed for the next delivery of nitric oxide. The data are shown below in Table 1.

Table 1, below, illustrates a data set collected in this Example. Variation in the breath sensitivity setting resulted in an increase in trigger threshold (measured in cm H₂O) from −1.0 at the least sensitive setting (1) to −0.1 at the most sensitive setting (10). In addition, the arming threshold (measured in cm H₂O) stayed constant at 0.1 from a sensitivity setting of 1 through a setting of 6, and decreased by 0.02 for each sensitivity setting thereafter through 10. This indicates that the most sensitive breath sensitivity setting allows breaths to be detected more accurately, which leads to more accurate pulsatile delivery of nitric oxide in a shorter window of time, i.e, earlier in the inspiratory part of the breath. Based on these data, additional tests were performed at sensitivity settings of 8 and 10.

TABLE 1
Breath Sensitivity and Trigger/Arming Thresholds

| | Breath Sensitivity | | | | | | | | | |
|---|---|---|---|---|---|---|---|---|---|---|
| | 1 | 2 | 3 | 4 | 5 | 6 | 7 | 8 | 9 | 10 |
| Trigger | −1.0 | −0.9 | −0.8 | −0.7 | −0.6 | −0.5 | −0.4 | −0.3 | −0.2 | −0.1 |
| Threshold | | | | | | | | | | |
| (cm H₂O) | | | | | | | | | | |
| Arming | +0.1 | +0.1 | +0.1 | +0.1 | +0.1 | +0.1 | +0.08 | +0.06 | +0.04 | +0.02 |
| Threshold | | | | | | | | | | |
| (cm H₂O) | | | | | | | | | | |

The conclusion is that a higher breath sensivity setting correlates to a lower trigger threshold and a higher arming threshold, which prepares the device to deliver short, precise pulses of nitric oxide over the therapy treatment course.

### Example 2: Testing a Device Against Various Breath Patterns

As discussed above, accurate and timely delivery of nitric oxide is critical to the present invention. In order to ensure that a device will deliver a precise dose of gas within a precise window of time, ten different breath patterns were tested using a mechanical lung and nose model. Ten different simulated breath patterns were analyzed, and the breath patterns had varying respiratory rate (8 to 36 bpm), tidal volume (316 to 912 ml), and Inspiration:Expiration (I/E) ratios (1:1 to 1:4). These variable breath patterns are patterns expected for subjects age 16 and up and are summarized in Table 2. Real world conditions were emulated to the extent possible.

TABLE 2
Summary of Breath Patterns Tested

| | | | Ideal | | | |
|---|---|---|---|---|---|---|
| | | | Body | Tidal | | |
| Respiratory | Male/ | Height | Weight | Volume | Inspiration | I:E |
| Rate (bpm) | Female | (cm) | (kg) | (mL) | Time (sec) | Ratio |
| 8 | F | 174 | 68.1 | 456 | 1.5 | 1:4 |
| 8 | M | 186 | 86.4 | 564 | 1.5 | 1:4 |
| 12 | F | 152 | 51.9 | 316 | 1.25 | 1:3 |
| 12 | M | 186 | 86.4 | 564 | 1.25 | 1:3 |
| 18 | F | 174 | 68.1 | 456 | 1.1 | 1:2 |
| 18 | M | 186 | 86.4 | 564 | 1.1 | 1:2 |
| 24 | F | 152 | 51.9 | 316 | 1.0 | 1:1.5 |
| 24 | F | 174 | 68.1 | 456 | 1.0 | 1:1.5 |
| 36 | F | 152 | 51.9 | 632 | 0.8 | 1:1 |
| 36 | F | 174 | 68.1 | 912 | 0.8 | 1:1 |

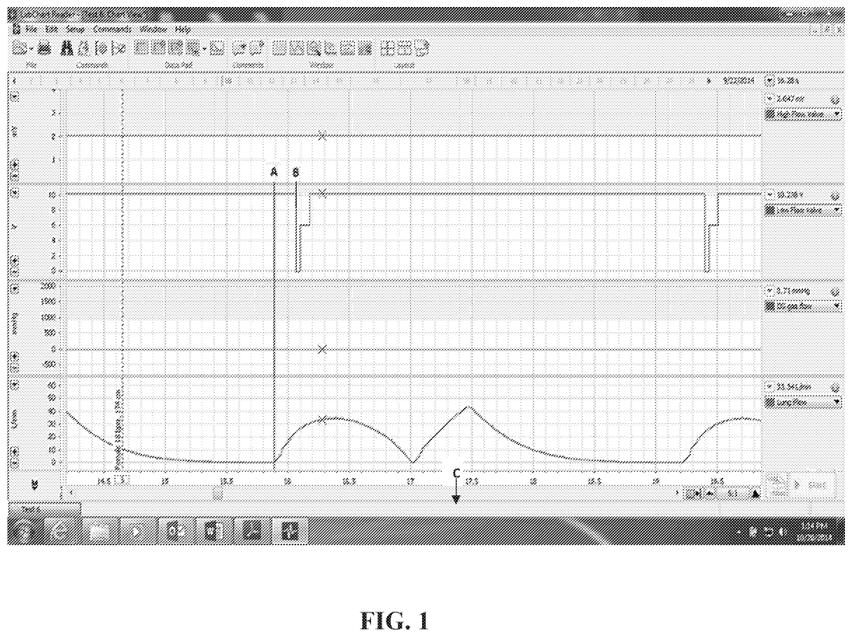

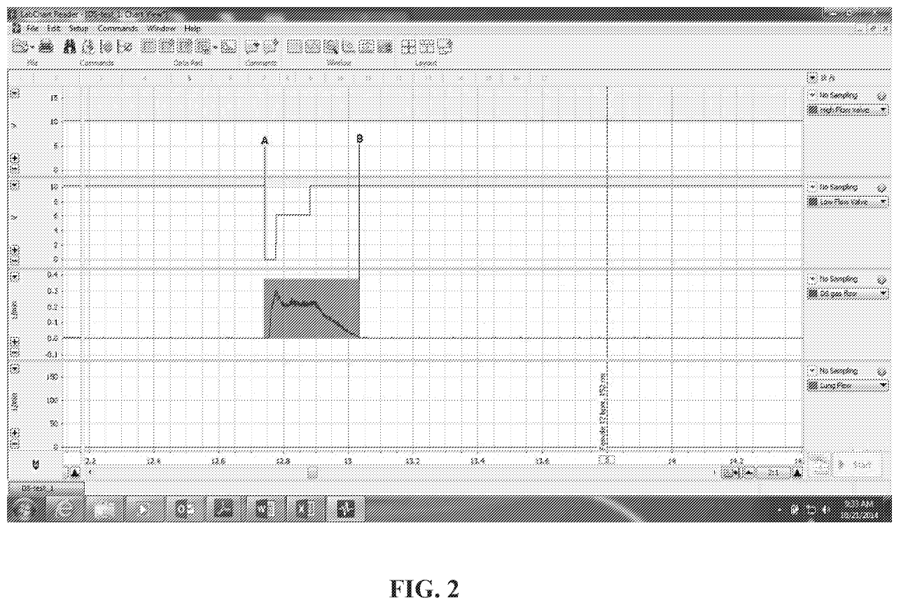

Two device embodiments were tested—Embodiment 1 was tested at sensitivity level 8 and sensitivity level 10, and the other device embodiment (Embodiment 2, which further includes a slope algorithm) was tested at sensitivity level 10. The investigation consisted of two parts. Part 1 measured the time delay between the initiation of the inspiratory breath and the onset of nitric oxide delivery using the 10 different simulated respiratory patterns. This time delay is measured using two data points—the time between initiation of inspiration (FIG. 1, Point A) and breath detection with concurrent opening of the delivery valve (FIG. 1, Point B). Part 2 measured the duration and volume of the delivered pulse covering the same breath patterns in Table 2. The time duration of the gas pulse is measured, from breath detection and concurrent opening of the delivery valve, which corresponds to the initiation of gas delivery, (FIG. 2, Point A) to the completion of the gas delivery (FIG. 2, Point B). The volume of the delivered pulse is measured by integration of the gas flow over the pulse duration. In addition, data from Part 1, measured time delay, and Part 2, measured pulse duration, are added to calculate the dose delivery time, sometimes referred to as “delivered pulse width”.

Part 1: Measuring Time Delay Between Initiation of Inspiration and Onset of NO Delivery.

This portion of the test was conducted at a dose of 75 ug/kg-IBW/hr with a drug concentration input of 6 mg/L (4880 ppm). This test was conducted using nitrogen only. The primary output for Part 1 is time duration between initiation of inspiration and valve opening/breath detection indication. Point A in FIG. 1 is the point where the lung air flow rises just above resting line. The time of valve opening is indicated as Point B in FIG. 1 and is displayed as a sudden voltage drop in the detector. The time interval between Point A and Point B is the valve time delay, or trigger delay, and is calculated for each breath pattern. The total inspiratory time corresponds to the interval from Point A to Point C (which is the end of inspiration).

Part 2: Measuring the Duration and Volume of the Delivered Pulse.

The same breath patterns were used in this part of the investigation. Doses of 10, 15, 30 and 75 ug/kg-IBW/hr were tested. The device was programmed for each dose, patient IBW, and respiratory rate (breaths per minute). The resulting pulsatile gas flow was determined by a flow meter. The pulse duration is the time between the point at which the valve opening was indicated, displayed as a sudden voltage drop in the detector, corresponding to Point A in FIG. 2, and the time at which the gas flow returns to baseline at Point B in FIG. 2. The volume of the delivered pulse is the integrated gas flow during the pulse duration. The pulse duration was added to the pulse delay from Part 1 to give the dose delivery time or “delivered pulse width.” FIG. 1. illustrates the results of Part 1. There are four panels shown in FIG. 1. The second and fourth panels show the breath detection which corresponds to the flow control valve operation and a representation of a breath pattern, respectively. Point A shows initiation of inspiration, Point B shows breath detection which corresponds to the opening of the flow valve, and Point C shows the end of inspiration. From this data, the time delay between points A and B can be calculated.

FIG. 2. illustrates the results of Part 2. There are four panels shown in FIG. 2. The second and third panels show the breath detection which corresponds to the flow control valve operation and a representation of the pulsatile gas flow, respectively. Point A shows breath detection which corresponds to the opening of the flow valve and Point B shows the end of pulsatile flow. From this data, the pulse duration between points A and B can be calculated.

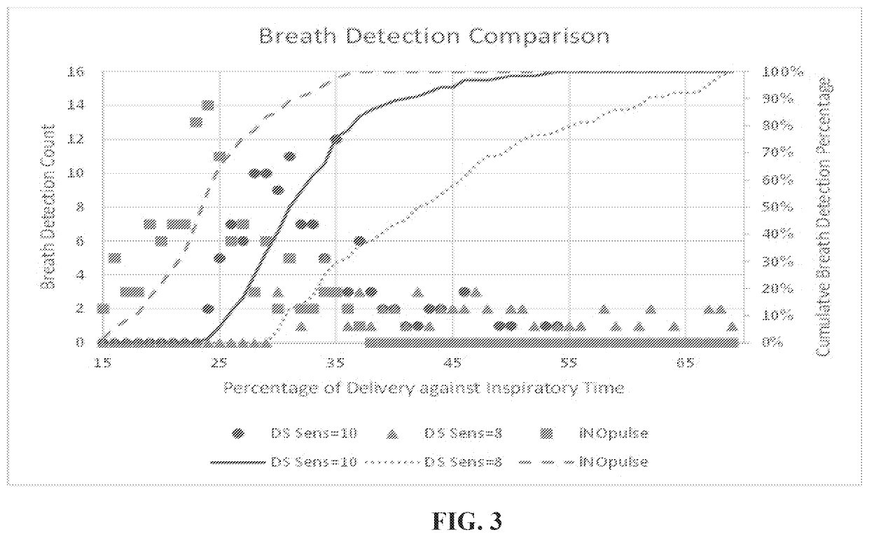

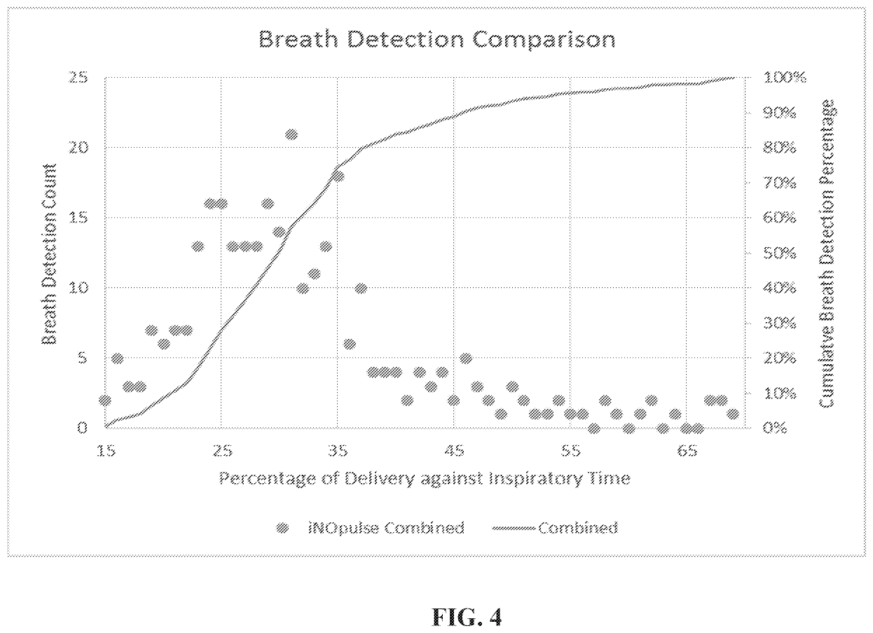

Table 3, below, summarizes the results depicted in FIG. 3 and FIG. 4.

| | % Delivery of NO Within Portion of Inspiratory Time | | |
|---|---|---|---|
| Device | First Third | First Half | First Two-thirds |
| Embodiment 1 | 17 | 77 | 95 |
| (Sensitivity 8) | | | |
| Embodiment | 62 | 98 | 100 |
| 1(Sensitivity 10) | | | |
| Embodiment 2 | 93 | 100 | 100 |
| Combined Data | 64 | 93 | 99 |

FIG. 3 depicts results for the breath detection count for each device listed in Table 3. The Embodiment 2, or the square/dotted line data in FIG. 3, illustrates that at least 93% of nitric oxide is delivered within the first third of the inspiratory portion of the breath. 100% of the nitric oxide is delivered within the first half of the inspiratory portion of the breath. Comparatively, for the Embodiment 1 at a sensitivity setting of 8, at least 17% of the nitric oxide is delivered within the first third of the inspiratory portion of the breath, at least 77% within the first half, and at least 95% within the first two-thirds of the inspiratory portion of the breath. The Embodiment 1 at a sensitivity setting of 10 showed results that at least 62% of nitric oxide is delivered within the first third of the inspiratory portion of the breath, at least 98% in the first half, and 100% in the first two-thirds of the inspiratory portion of the breath. FIG. 4 depicts the combined data curve for all three tests.

This data concludes that lower doses of nitric oxide are needed over the course of a single treatment because more nitric oxide is being more precisely delivered with each pulse over a shorter period of time during the course of treatment. Lower doses of nitric oxide may lead to use of less drug overall, and also may lead to less risk of detrimental side effects.

We claim:

1. A method for delivery of a dose of nitric oxide to a patient in need, said method comprising:
a) Detecting a breath pattern in said patient including a total inspiratory time using a device comprising a breath sensitivity control;
b) Correlating the breath pattern with an algorithm to calculate the timing of administration of the dose of nitric oxide; and
c) Delivering the dose of nitric oxide to said patient in a pulsatile manner over a portion of the total inspiratory time.

2. The method of claim 1, wherein delivery of the dose of nitric oxide occurs within the first third of the total inspiratory time.

3. The method of claim 1, wherein delivery of the dose of nitric oxide occurs within the first two-thirds of the total inspiratory time.

4. The method of claim 1, wherein delivery of the dose of nitric oxide occurs within the first half of the total inspiratory time.

5. The method of claim 1, wherein delivery of at least fifty percent of the dose of nitric oxide occurs within the first third of the total inspiratory time.

6. The method of claim 1, wherein delivery of at least ninety percent of the dose of nitric oxide occurs within the first two-thirds of the total inspiratory time.

7. The method of claim 1, wherein delivery of at least 70 percent of the dose of nitric oxide occurs within the first half of the total inspiratory time.

8. The method of claim 1, wherein the nitric oxide is delivered in a series of pulses over a period of time.

9. The method of claim 1, wherein the breath sensitivity control is adjustable.

10. The method of claim 1, wherein the breath sensitivity control is fixed.

11. The method of claim 1, wherein the nitric oxide delivery has an antimicrobial effect.

12. A method for treating a cardiopulmonary disease in a patient said method comprising:
a) Detecting a breath pattern in said patient using a device comprising a breath sensitivity control, said breath pattern having a total inspiratory time and a total expiratory time;
b) Correlating the breath pattern with an algorithm to calculate the timing of administration of a dose of nitric oxide;
c) Delivering said dose of nitric oxide to said patient in a pulsatile manner over a portion of the total inspiratory time for a period of time required for a therapeutically effective amount of nitric oxide to be delivered to said patient.

13. The method of claim 12, wherein the cardiopulmonary disease is selected from the group consisting of idiopathic pulmonary fibrosis (IPF), pulmonary hypertension or pulmonary arterial hypertension (PH or PAH), Group I-V pulmonary hypertension, chronic obstructive pulmonary disease (COPD), combined pulmonary fibrosis and emphysema (CPFE), emphysema, interstitial lung disease (ILD), chronic thromboembolic pulmonary hypertension (CTEPH), chronic high altitude sickness, or other lung disease.

14. The method of claim 12, wherein the cardiopulmonary disease is Group I-V pulmonary hypertension (PH).

15. The method of claim 1, wherein less than 10% of poorly ventilated (a) areas of the lung or (b) alveoli are exposed to a nitric oxide.

16. A programmable device for delivering a dose of nitric oxide to a patient in need, the device comprising:
a) A delivery portion;
b) A drug cartridge comprising nitric oxide;
c) An oxygen source;
d) A breath sensitivity portion to detect a breath pattern in said patient comprising a breath sensitivity setting;
e) A breath detection algorithm for determining the dose of nitric oxide; and
f) A portion for administering the dose of nitric oxide to the patient through a series of pulses.

17. The device of claim 16, wherein the breath sensitivity is fixed or adjustable from a value of least sensitive to a value of most sensitive.

18. The device of claim 16, wherein the breath sensitivity setting is fixed at most sensitive.

19. The device of claim 16, wherein the drug cartridge is replaceable.

20. The device of claim 16, wherein the nasal delivery portion is selected from the group consisting of a nasal cannula, a face mask, an atomizer, and a nasal inhaler.

21. A breath detection algorithm for determining the timing for delivering a pulse of nitric oxide to patient, wherein the algorithm uses a threshold sensitivity and a slope algorithm, wherein the slope algorithm detects breath when the rate of pressure drop reaches a minimum threshold.

22. A method of administering gas to a subject comprising administering a pulse dose of a first gas to a subject upon detection of an inspiration by the subject.

23. The method of claim 22, wherein greater than 50% of the total number of pulse doses administered to the patient are administered during the first half of a total inspiration time of the detected inspiration.

24. The method of claim 22, further comprising administering a second gas to the subject, wherein administration of the pulse dose of the first gas minimizes interference with a pressure sensor associated with the administration of the second gas.

25. The method of claim 24, wherein the first gas is nitric oxide and the second gas is oxygen.

26. The method of claim 1, wherein the dose of nitric oxide is a therapeutically effective dose.
